(19) United States
(12) Patent Application Publication
(10) Pub. No.: US 2013/0150718 A1
(43) Pub. Date: Jun. 13, 2013

(54) ULTRASOUND IMAGING SYSTEM AND METHOD FOR IMAGING AN ENDOMETRIUM

(71) Applicants: Adam J. Dixon, Charlottesville, VA (US); Michael J. Washburn, Wauwatosa, WI (US)

(72) Inventors: Adam J. Dixon, Charlottesville, VA (US); Michael J. Washburn, Wauwatosa, WI (US)

(73) Assignee: GENERAL ELECTRIC COMPANY, Schenectady, NY (US)

(21) Appl. No.: 13/313,927

(22) Filed: Dec. 7, 2011

(51) Int. Cl. A61B 8/12

(52) U.S. Cl. 600/443

(57) ABSTRACT

An ultrasound imaging system and method for ultrasound imaging. The ultrasound imaging system includes a probe, a display device and a processing unit in electronic communication with the probe and the display device. The processing unit is configured to identify and display an image of an endometrium. The method includes acquiring ultrasound data, selecting a range of depths, acquiring 3D ultrasound data from within the range of depths, calculating an average image, identifying an image that is the closest fit to the average image, and displaying the image.

### FIELD OF THE INVENTION

This disclosure relates generally to an ultrasound imaging system and a method for obtaining an image of a patient's endometrium.

### BACKGROUND OF THE INVENTION

3D ultrasound has emerged as a preferred modality for acquiring 3D data of uterine anatomy due to its high level of availability and lack of ionizing radiation. 3D endovaginal probes have emerged as the standard of care for uterine imaging due to their ability to acquire renderings of both longitudinal and transverse planes in the volume. In particular, renderings of the coronal plane, or C-plane, which includes planes that are generally parallel to the transducer array, are of particular interest when visualizing the endometrium. In order to make many diagnoses of uterine pathologies, it is desired to view an image of the endometrium. However, current workflows require clinicians to acquire 3D ultrasound data and manually search through the volume for the best images of the endometrium.

Conventional image processing techniques to identify the endometrium have had limited clinical success primarily due to the fact that the morphology of the endometrium varies widely. Since endometria come in a variety of different shapes and orientations, image-based segmentation techniques have not proven to be a reliable method of obtaining clinically useful images of the endometrium. Additionally, the intensity of the endometrium with respect to its surroundings may also vary greatly. This makes automatic thresholding techniques based on intensity of limited use.

According to conventional workflow, the clinician is required to acquire a 3D volume of ultrasound data and then manually locate the most-appropriate C-plane of the endometrium within the volume. At the very least, this method requires the clinician to manually sort through a number of images before selecting the most appropriate one. However, most of the time the endometrium is not aligned exactly with the C-plane. For cases like these, the clinician is also required to adjust the tilt of the C-plane in order to capture the best image of the endometrium. On conventional ultrasound imaging systems, the clinician may be required to manipulate multiple rotaries, touch panel buttons, and physical buttons on the front panel in order to locate the best image of the endometrium. Even the most experienced ultrasound clinicians may become disoriented after performing multiple manipulations on the volume according to conventional techniques

For these and other reasons an improved method and system for obtaining ultrasound images of the endometrium is desired.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes acquiring ultrasound data with a probe, rendering a first image from the ultrasound data and selecting a range of depths from the first image, wherein the range of depths includes an endometrium. The method includes acquiring 3D ultrasound data with the probe and rendering a plurality of images from the 3D ultrasound data, wherein each of the plurality of images intersects the first image within the selected range of depths. The method includes calculating an average image from the plurality of images, identifying one of the plurality of images that is the closest fit to the average image and displaying the one of the plurality of images that is the closest fit to the average image, wherein the one of the plurality of images includes the endometrium.

In another embodiment, a method of ultrasound imaging includes acquiring ultrasound data with a probe, rendering a first image from the ultrasound data and selecting a range of depths from the first image, wherein the range of depths includes the endometrium. The method includes acquiring 3D ultrasound data with the probe and generating a projection from the 3D ultrasound data only from within the selected range of depths. The method includes identifying a curved plane from the 3D ultrasound data that fits the projection and displaying an image based on the curved plane.

In another embodiment, an ultrasound imaging system includes a probe adapted to scan a volume of interest, a display device and a processing unit in electronic communication with the probe and the display device. The processing unit is configured to control the probe to acquire ultrasound data including an endometrium, render a first image from the ultrasound data, display the first image on the display device, and acquire 3D ultrasound data including a range of depths selected through a user input. The processing unit is configured to render a plurality of images from the 3D ultrasound data, wherein each of the plurality of images intersects the first image within the selected range of depths. The processing unit is configured to calculate an average image from the plurality of images and identify one of the plurality of images that is the closest fit to the average image. The processing unit is also configured to display the one of the plurality of images on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of images representing some of the various endometrium morphologies;

FIG. 3 is a schematic representation of a 2D array probe in accordance with an embodiment;

FIG. 4 is a flow chart in accordance with an embodiment;

FIG. 5 is a schematic representation of an image of an endometrium with a range gate in accordance with an embodiment;

FIG. 6 is a flow chart in accordance with an embodiment; and

FIG. 7 is a schematic representation of the steps of a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

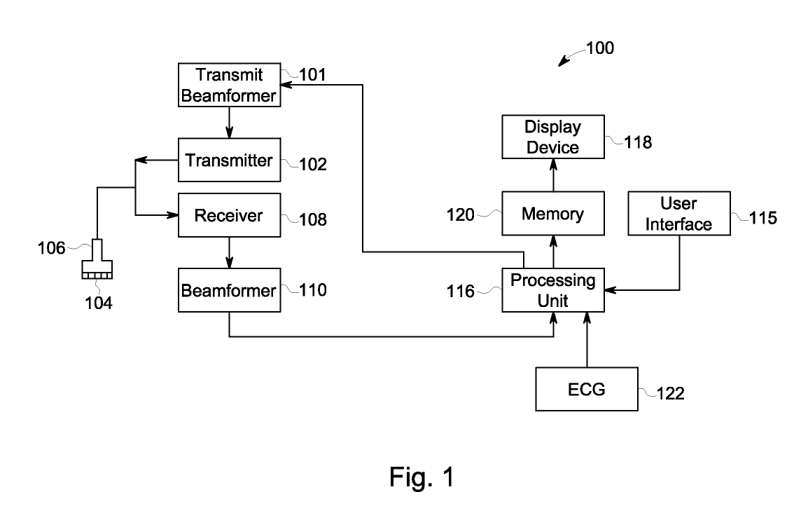

FIG. 1 is a schematic diagram of an ultrasound imaging system 100. The ultrasound imaging system 100 includes a transmit beamformer 101 and a transmitter 102 that drive transducer elements 104 within a probe 106 to emit pulsed ultrasonic signals into a body (not shown). The probe 106 may be a 2D array probe according to an embodiment. According to other embodiment, the probe 106 may be any other type of probe capable of acquiring 3D ultrasound data, including a mechanical 3D ultrasound probe. A variety of geometries of probes and transducer elements may be used. The pulsed ultrasonic signals are back-scattered from structures in the body, like blood cells or muscular tissue, to produce echoes that return to the transducer elements 104. The echoes are converted into electrical signals, or ultrasound data, by the transducer elements 104 and the electrical signals are received by a receiver 108. According to some embodiments, the probe 106 may contain electronic circuitry to do all or part of the transmit and/or the receive beamforming. For example, all or part of the transmit beamformer 101, the transmitter 102, the receiver 108 and the beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. The electrical signals representing the received echoes are passed through a beamformer 110 that outputs ultrasound data. A user interface 115 may be used to control operation of the ultrasound imaging system 100, including, to control the input of patient data, to change a scanning or display parameter, and the like.

The ultrasound imaging system 100 also includes a processing unit 116 to control the transmit beamformer 101, the transmitter 102, the receiver 108 and the beamformer 110. The processing unit 116 is in electronic communication with the probe. The processing unit 116 may control the probe 106 to acquire 3D ultrasound data. The processing unit 116 controls which of the transducer elements 104 are active and the shape of a beam emitted from the probe 106. The processing unit 116 is also in electronic communication with a display device 118, and the processing unit 116 may process the data into images for display on the display device 118. For purposes of this disclosure, the term “electronic communication” may be defined to include both wired and wireless connections. The processing unit 116 may comprise a central processing unit (CPU) according to an embodiment. According to other embodiments, the processing unit 116 may comprise other electronic components capable of carrying out processing functions, such as a digital signal processing unit, a field-programmable gate array (FPGA) or a graphic board. According to other embodiments, the processing unit 116 may comprise multiple electronic components capable of carrying out processing functions. For example, the processing unit 116 may comprise two or more electronic components selected from a list of electronic components including: a central processing unit, a digital signal processing unit, a field-programmable gate array, and a graphic board. According to another embodiment, the processing unit 116 may also include a complex demodulator (not shown) that demodulates the RF data and generates raw data. In another embodiment the demodulation can be carried out earlier in the processing chain. The processing unit 116 is adapted to perform one or more processing operations according to a plurality of selectable ultrasound modalities on the data. The ultrasound data may be processed in real-time during a scanning session as the echo signals are received. For the purposes of this disclosure, the term “real-time” is defined to include a procedure that is performed without any intentional delay. For example, an embodiment may acquire and display images with a real-time frame-rate of 7-20 frames/sec. However, it should be understood that the real-time frame rate may be dependent on the length of time that it takes to acquire each frame of ultrasound data for display. Accordingly, when acquiring a relatively large volume of data, the real-time frame rate may be slower. Thus, some embodiments may have real-time frame-rates that are considerably faster than 20 frames/sec while other embodiments may have real-time frame-rates slower than 7 frames/sec. The ultrasound information may be stored temporarily in a buffer (not shown) during a scanning session and processed in less than real-time in a live or off-line operation. Some embodiments of the invention may include multiple processing units (not shown) to handle the processing tasks. For example, a first processing unit may be utilized to demodulate and decimate the RF signal while a second processing unit may be used to further process the data prior to displaying an image. It should be appreciated that other embodiments may use a different arrangement of processing units.

The ultrasound imaging system 100 may continuously acquire data at a frame-rate of, for example, 10 Hz to 30 Hz. Images generated from the data may be refreshed at a similar frame rate. Other embodiments may acquire and display data at different rates. For example, some embodiments may acquire data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store at least several seconds worth of frames of ultrasound data. The frames of data are stored in a manner to facilitate retrieval thereof according to its order or time of acquisition. The memory 120 may comprise any known data storage medium. There is an ECG 122 attached to the processing unit 116 of the ultrasound imaging system 100 shown in FIG. 1. The ECG may be connected to the patient and provides cardiac data from the patient to the processing unit 116 for use during the acquisition of cardiac gated ultrasound data.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processing unit 116 (e.g., B-mode, Color Doppler, M-mode, Color M-mode, spectral Doppler, TVI, strain, strain rate, and the like) to form 2D or 3D data. For example, one or more modules may generate B-mode, color Doppler, M-mode, color M-mode, spectral Doppler, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processing unit module may be provided that reads the image frames from a memory and displays the image frames in real time while a procedure is being carried out on a patient. A video processing unit module may store the image frames in an image memory, from which the images are read and displayed.

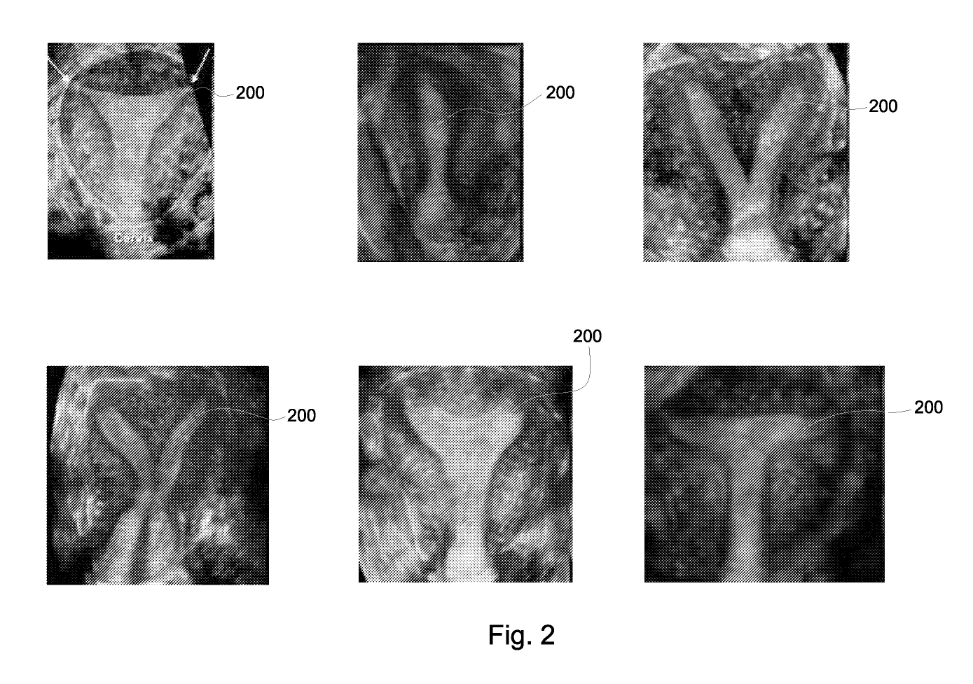

FIG. 2 is representation of six different exemplary endometrium morphologies as acquired with an ultrasound imaging system such as ultrasound imaging system 100. The endometrium 200 is labeled in each of the images. FIG. 2 shows the wide range of shapes, sizes and orientations that endometria may exhibit in different patients.

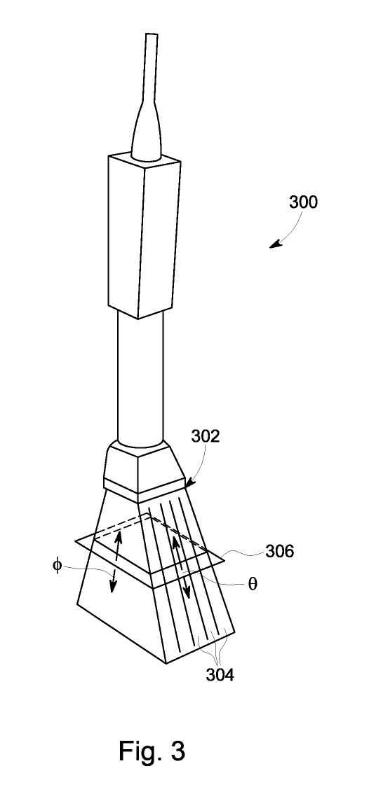

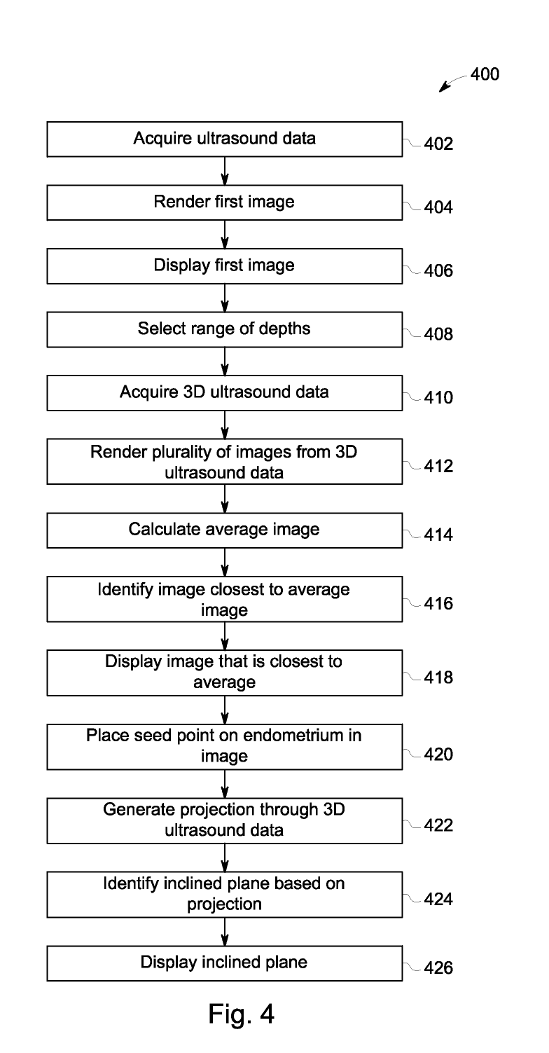

FIG. 3 is a schematic representation of a 2D array probe 300. The 2D array probe 300 is a transvaginal volume probe according to an embodiment. The 2D array probe 300 may be connected to the ultrasound imaging system 100 in place of probe 106. The 2D array probe 300 includes an array 302 of transducer elements arranged in a 2D matrix. The 2D array probe 300 may be controlled to acquire either a volume of data or a plane of data depending upon how the individual transducer elements are controlled. According to an exemplary embodiment, the 2D array probe 300 may be controlled to acquire 3D ultrasound data by acquiring multiple planes 304 of data. By combining data from each of the planes 304, the 2D array probe 300 acquires data for the entire volume. Mechanical 3D ultrasound probes may acquire a volume of data in the same manner as that described hereinabove with respect to the 2D array probe 300 of FIG. 3. Mechanical 3D ultrasound probes may acquire a volume of data in the same manner as that described hereinabove with respect to the 2D array probe 300 of FIG. 3. According to other embodiments, a volume of data may be acquired through the acquisition of a plurality of planes that are not parallel to each other. For example, according to an embodiment, a rotating or rocking transducer array may be used to acquire planes disposed at multiple different angles. A schematic representation of a C-plane 306 is shown. The term “C-plane” is defined to include planes that are substantially parallel to the transducer 302. Referring to FIG. 4, for purposes of this disclosure, the term “C-plane” is also defined to include planes passing through the acquired volume that do not intersect with the transducer array 304. For example, arrows show how the C-plane 306 may be tilted in a ⊖ direction or a Φ direction, or in a combination of the ⊖ direction and the Φ direction. Due to physiological constraints when imaging endometria with an endovaginal probe, the image of the endometrium is most often a C-plane image.

FIG. 4 is a flow chart showing steps of a method 400 in accordance with an embodiment. The individual blocks represent steps that may be performed in accordance with the method 400. The method 400 may be implemented by a processing unit, such as the processing unit 116 shown in FIG. 1, according to an embodiment. The technical effect of the method 400 is the selection and display of an image of the endometrium.

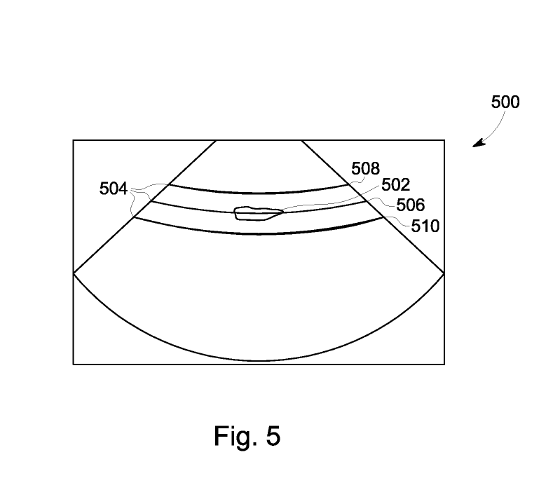

FIG. 5 is a schematic representation of an image of an endometrium in accordance with an embodiment.

Referring to both FIGS. 4 and 5, at step 402, the processing unit 116 (shown in FIG. 1) controls the acquisition of ultrasound data. The ultrasound data may be 2D ultrasound data or the ultrasound data may be 3D ultrasound data. According to an embodiment, the ultrasound data may be acquired with a probe such as the probe 300 (shown in FIG. 3). At step 404, a first image is rendered from the ultrasound data. FIG. 5 is a schematic representation of an image of an endometrium 500 in accordance with an embodiment. The first image from the method 400 may be an image similar to the image of the endometrium 500. The first image may be an image of a plane that would intersect the transducer array of the probe at the time of acquisition according to an embodiment. According to embodiments where the ultrasound data acquired at step 402 is 2D ultrasound data, the method 400 may render an image of the whole plane captured by the 2D ultrasound data. Referring back to FIG. 4, at step 406, the first image is displayed on a display device such as the display device 118 (shown in FIG. 1).

Next, at step 408, the clinician selects a range of depths from the first image including the endometrium. According to an embodiment, the clinician may use a range gate to select the range of depths. Referring to FIG. 5, a range gate 504 includes a mid-depth indicator 506, an upper depth indicator 508, and a lower depth indicator 510. The clinician may adjust the mid-depth indicator 506, the upper depth indicator 508, and the lower depth indicator 510 independently, or the mid-depth indicator 506, the upper depth indicator 508, and the lower depth indicator 510 may be linked so that movement to one indicator affects the location of one or both of the other indicators. For example, the clinician may be able to position the mid-depth indicator 506 on approximately the middle of the endometrium, and then the clinician may move one of either the upper depth indicator 508 and the lower depth indicator 510. The processing unit 116 (shown in FIG. 1) may automatically position the other of the upper depth indicator 508 or the lower depth indicator 510 an equal distance away from the mid-depth indicator 506. According to an embodiment, the clinician may position the range gate 504 so that most of the endometrium is within the upper depth indicator 508 and the lower depth indicator 510. It should be appreciated that the range of depths may be selected according to different techniques in other examples. For example, the clinician may numerically enter the range of depths where the endometrium is visible. According to other embodiments, the processing unit 116 may automatically select the range of depths. For example, the processing unit 116 may select the range of depths to include the ranges of depths that are typical for the endometrium. Or, the processing unit 116 may automatically select the range of depths through techniques including image processing and/or the comparison of the current image to one or more images in an image database. If the processing unit 116 identifies the appropriate range of depths for the endometrium, then no further action is required by the clinician. However, if it is necessary to make any adjustments to the range of depths, then the clinician may manually adjust the range of depths through a manual technique, including any of the techniques described above. The clinician may also use any other type of user interface or technique to input the range of depths of the endometrium.

At step 410, 3D ultrasound data of the endometrium is acquired. The 3D ultrasound data includes ultrasound data from within the range of depths selected in step 408. If speed of acquisition is of concern, then the 3D ultrasound data may be acquired only from within the selected range of depths. However, according to other embodiments, 3D ultrasound data including additional depths outside the range of depths may also be acquired. According to other embodiments, steps 408 and 410 may be switched; that is, the 3D ultrasound data may be acquired before the range of depths is selected. However, as will be described hereinafter, according to an embodiment, only the 3D ultrasound data from within the range of depths will be used for calculating an average image.

Next, at step 412, a plurality of images are rendered from the 3D ultrasound data acquired at step 410. According to an embodiment, each of the images may be a C-plane image and each of the C-plane images may be parallel to each other. According to an exemplary embodiment, each of the plurality of images may be substantially parallel to the transducer array of the probe used to acquire the 3D ultrasound data. The plurality of images rendered by the processing unit 116 at step 412 may include an image at each possible depth within the range of depths or the plurality of images may include only a subsampling of all the possible images within the range of depths. It may be advantageous to only render a subsampling of the images within the range of depths in order to implement step 412 more quickly.

At step 414, an average image is calculated from the plurality of images. The average image may be a median image, a mean image, or any other type of mathematical average that is representative of the plurality of images as a group. According to an embodiment, the median image may be calculated by determining a median sample intensity value along each of a plurality of perpendicular vectors passing through the plurality of images. For example, a median value may be calculated along a perpendicular vector for each pixel in the median image. This way, the median image represents an average of the plurality of images. Next, at step 416, a processing unit, such as the processing unit 116, identifies which of the plurality of images rendered at step 412 is the closest fit to the average image. Back at step 408, the clinician had selected a range of depths including the endometrium. According to an embodiment, the clinician may select the range of depths so that upper range limit is close to the expected top of the endometrium and the lower range limit is close to the expected bottom of the endometrium. Ideally, most or all of the images rendered from the 3D ultrasound data within the selected range of depths will include at least a portion of the endometrium. The processing unit 116 (shown in FIG. 1) implements an algorithm that searches each of the plurality of images to identify which of the plurality of images has the best signature of the endometrium. During step 412, the processing unit 116 determines which of the plurality of images is closest to the average image. Based on the assumption that the endometrium is present in the majority of the images within the selected range of depths, the endometrium should be strongly represented in the average image.

The processing unit 116 may identify the image with the closest fit to the average image by using a similarity metric to compare the image to the average image. According to an exemplary embodiment, the processing unit 116 may use mean-squared error as the similarity metric. For example, the processing unit 116 may calculate the mean-squared error of each of the plurality of images rendered at step 412 with respect to the average image. The processing unit 116 may then select the image with the lowest mean-squared error as the closest fit to the average image. The image with the lowest mean-squared error may be selected as a representative C-plane view of the endometrium. At step 418, the image is displayed on a display such as display device 118. According to some embodiments, the method 400 may stop after step 418. According to other embodiments, other types of similarity metrics may be used. For example, sums of squared errors and correlations are non-limiting examples of other similarity metrics that may be used. According to some embodiments, a refinement of the image may be desired. According to these embodiments, the method 400 continues with step 420, where the clinician places a seed point on the endometrium within the image displayed at step 418. The clinician may place the seed point approximately in the center of the endometrium, although the algorithm will work as long as the clinician accurately places the seed point on the endometrium. In other embodiments, the seed point may be placed on the endometrium automatically by the processing unit 116. For example, the processing unit 116 may plot a histogram based on the similarity of the central portion of the image to the average image. Then, the processing unit 116 could identify a seed point by calculating an average location of a number of samples or pixels that are closest to the peak of the histogram.

One of the challenges involved with segmenting the endometrium from ultrasound data is that the endometrium may have a higher intensity than surrounding tissue or a lower intensity than surrounding tissue. However, by having the user place a seed point on the endometrium, it is possible for an algorithm to accurately determine the intensity of the endometrium with respect to the surrounding tissue. Next, at step 422, the processing unit 116 generates a projection through the 3D ultrasound data. If the endometrium has a higher intensity than the surrounding tissue, then the method 400 may generate a maximum intensity projection (MIP) through 3D ultrasound data. If the endometrium has a lower intensity than the surrounding tissue, then the method 400 may generate a minimum intensity projection (MinIP) through the 3D ultrasound data. According to an embodiment, the method 400 generates the projection based on the 3D ultrasound data only within the selected range of depths identified by the clinician at step 408. Since the projection is generated based on the 3D ultrasound data within the range of depths identified by the user as most likely to contain the endometrium, and since the clinician placed a seed point in the endometrium during step 420, it is likely that the projection will accurately capture the morphology of a particular patient's endometrium.

Next, at step 424, the method 400 identifies an inclined plane within the 3D ultrasound data that is closest to the projection generated at step 422. For the purposes of this disclosure, the term “inclined plane” is defined to include a plane that is tilted or angled with respect to the plane defined by the transducer array. According to an embodiment, the algorithm may compare renderings generated from the 3D ultrasound data at a plurality of different angles of Φ and ⊖ in order to identify an inclined plane that is most similar to the projection from step 422. For example, the processing unit may compare renderings across a range of angle for Φ and a range of angles for ⊖. The arrows in FIG. 3 schematically illustrates how changes in both ⊖ and Φ affect the angle of the c-plane with respect to the transducer 300. The processing unit 116 may then use the plane with the combination of angles in the Φ direction and the ⊖ direction that results in the closest fit to the projection from step 422. Mean-squared error may be used to compare renderings of the various planes to the projection or other comparison techniques, including cross-correlation, may be used. At step 426, a rendering of the inclined plane identified at step 424 is displayed on the display device 118 (shown in FIG. 1).

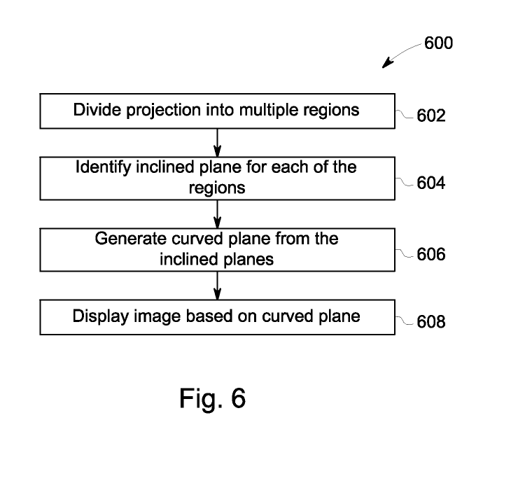

FIG. 6 is a flow-chart illustrating a method 600 that may replace steps 424 and 426 of the method 400 according to an embodiment. The technical effect of the method 600 is the identification of the curved plane within the 3D ultrasound data.

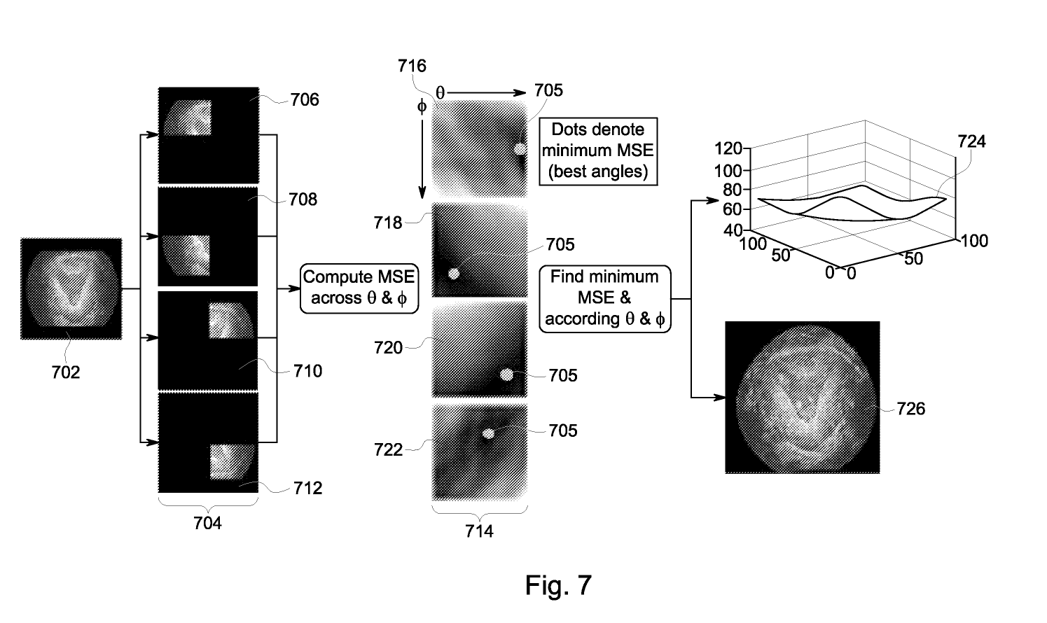

FIG. 7 is a schematic representation of the steps of the method 600 in accordance with an embodiment.

As discussed previously, the morphology of the endometrium may vary significantly between patients. From a clinical perspective, the best view of the endometrium may not always lie within a single flat plane. For example, if the overall shape of the endometrium is curved or s-shaped, it may be desirable to generate an image of the endometrium based on a curved plane. Referring to FIGS. 6 and 7, at step 602, a projection 702, hereinafter MinIP image 702, from step 422 of the method 400 may be divided into a plurality of regions 704. For example, the MinIP image 702 may be divided into a first region 706, a second region 708, a third region 710, and a fourth region 712 according to an embodiment. Hereinafter, the method 600 will be described according to an exemplary embodiment using four rectangular regions, but it should be appreciated by those skilled in the art that other embodiments may use a different size, shape and/or number of regions. At step 604, the processing unit 116 identifies an inclined plane for each of the four regions. For example, the four regions 704 may be rectangular quadrants that each share a common point. The 3D ultrasound data may also be divided into 4 sub-volumes. Each of the sub-volumes corresponds to one of the four regions. Each of the sub-volumes may be a box-shaped volume according to an embodiment. For example, a first sub-volume may include the anatomy shown in the first region 706, a second sub-volume may include the anatomy shown in the second region 708, a third sub-volume may include the anatomy shown in the third region 710, and a fourth sub-volume may include the anatomy shown in the fourth region 712. Starting with the plane identified during step 416, the processing unit 116 (shown in FIG. 1) may identify a plane within each of the sub-volumes that is the most similar to the corresponding region of the MinIP image 702. Mean-squared error may be used to compare each of the various planes within each of the sub-volumes to the corresponding regions of the MinIP image 702 or other comparison techniques, including cross-correlation, may also be used. Plots 714 showing the mean-squared error across angles of ⊖ and Φ for each of the sub-volumes compared to each of the corresponding regions 704. Dots 705 are included on each of the plots 714 to indicate the combination of ⊖ and Φ that results in the lowest mean-squared error. Since each of the dots represents a unique combination of inclinations in a ⊖ direction and Φ direction, each of the dots 705 identifies a plane with the lowest mean-squared error compared to the correspond region 704 of the MinIP image 702.

Next, at step 606 the processing unit 116 may combine the four planes into a single curved plane such as the curved plane 724. The curved plane 724 may be generated so that the contours of the curved plane 724 flow smoothly from the planes in the various sub-volumes or the curved plane may be “coarser” and include four discrete planes that do not smoothly flow from one plane to the next. The curved plane 724 represents the plane through the volume of 3D ultrasound data from which an image may be generated. The processing unit 116 may display an image based on the curved plane at step 608. For example, the processing unit 116 may display an image of the curved plane, such as image 726, or the processing unit 116 may display images of one or more flat planes that have been fit to the curved plane. For example, it may be easier to edit and/or understand the image if flat planes derived from the curved planes are displayed. The advantage of generating a curved plane depends upon the patient's anatomy and the details of the 3D ultrasound data. For situations where the best image of the endometrium is represented by a curved plane, it is possible to obtain a better final image of the endometrium by first fitting a curved plane to the average image and then fitting a flat plane to the curved plane. For most situations, generating a curved plane from the 3D ultrasound data before generating a flat plane should result in the selection of a flat plane that is a better fit to the average image.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
acquiring ultrasound data with a probe;
rendering a first image from the ultrasound data;
selecting a range of depths from the first image, wherein the range of depths includes an endometrium;
acquiring 3D ultrasound data with the probe;
rendering a plurality of images from the 3D ultrasound data, wherein each of the plurality of images intersects the first image within the selected range of depths;
calculating an average image from the plurality of images;
identifying one of the plurality of images that is the closest fit to the average image; and
displaying the one of the plurality of images that is the closest fit to the average image, wherein the one of the plurality of images comprises the endometrium.

2. The method of claim 1, wherein said calculating the average image comprises calculating a median image from the plurality of images.

3. The method of claim 1, wherein said calculating the average image comprises calculating a mean image from the plurality of images.

4. The method of claim 1, wherein said identifying the one of the plurality of images that is the closest fit to the average image comprises using a similarity metric to compare each of the plurality of images to the average image.

5. The method of claim 4, further comprising generating a projection through a portion of the 3D ultrasound data within the selected range of depths.

6. The method of claim 5, wherein said generating the projection comprises generating one of a maximum intensity projection and a minimum intensity projection.

7. The method of claim 6, further comprising identifying an inclined plane in the 3D ultrasound data that is the closest fit to the projection.

8. The method of claim 7, further comprising displaying a second image, wherein the second image comprises an image generated from the 3D ultrasound data at the location of the inclined plane.

9. The method of claim 1, wherein each of the plurality of images comprises a C-plane image.

10. The method of claim 9, wherein the C-plane images are perpendicular to the first image.

11. A method of ultrasound imaging comprising:
acquiring ultrasound data with a probe;
rendering a first image from the ultrasound data;
selecting a range of depths from the first image, wherein the range of depths includes the endometrium;
acquiring 3D ultrasound data with the probe;
generating a projection from the 3D ultrasound data, wherein the projection is generated only from 3D ultrasound data within the selected range of depths;
identifying a curved plane from the 3D ultrasound data that fits the projection; and
displaying an image based on the curved plane.

12. The method of claim 11, wherein said identifying the curved plane comprises dividing the projection into a plurality of regions and dividing the 3D ultrasound data into a plurality of sub-volumes, where each of the regions corresponds to a unique one of the sub-volumes.

13. The method of claim 12, wherein said identifying the curved plane further comprises identifying an inclined plane for each of the sub-volumes that is the closest fit to the corresponding region of the projection.

14. The method of claim 13, wherein said displaying the image comprises displaying a flat representation based on the curved plane.

15. An ultrasound imaging system comprising:
a probe adapted to scan a volume of interest;
a display device; and
a processing unit in electronic communication with the probe and the display device, wherein the processing unit is configured to:
control the probe to acquire ultrasound data including an endometrium;
render a first image from the ultrasound data;
display the first image on the display device;
acquire 3D ultrasound data including a range of depths selected through a user input;
render a plurality of images from the 3D ultrasound data, wherein each of the plurality of images intersects the first image within the selected range of depths;
calculate an average image from the plurality of images;
identify one of the plurality of images that is the closest fit to the average image; and
display the one of the plurality of images on the display device.

16. The ultrasound imaging system of claim 15, wherein the processing unit is configured to calculate the average image by identifying a median image of the plurality of images.

17. The ultrasound imaging system of claim 15, wherein the processing unit is configured to calculate the average image by calculating a mean image of the plurality of images.

18. The ultrasound imaging system of claim 15, wherein ultrasound probe comprises a 2D array probe.

19. The ultrasound imaging system of claim 15, wherein the processing unit is further configured to generate a projection through a portion of the 3D ultrasound data within the range of depths.

20. The ultrasound imaging system of claim 19, wherein the processing unit is further configured to identify an inclined plane through the 3D ultrasound data that is closest to the projection.

21. The ultrasound imaging system of claim 20, wherein the processing unit is configured to display a second image on the display device, wherein the second image comprises an image of the inclined plane.
